(19) United States
(12) Patent Application Publication
(10) Pub. No.: US 2022/0040418 A1
(43) Pub. Date: Feb. 10, 2022

(54) A DOSING REGULATOR AND RECOMMENDATIONS ENGINE

(71) Applicant: BANKSIDE INTERNATIONAL LTD., Devizes (GB)

(72) Inventors: Kyoungwoo Kim Blick, London (GB); Stephen Anthony Sayers, Weston Turville (GB)

(21) Appl. No.: 17/276,096

(22) PCT Filed: Sep. 18, 2019
(86) PCT No.: PCT/EP2019/075057

§ 371 Date: Mar. 12, 2021

(30) Foreign Application Priority Data
Sep. 19, 2018 (GB) 1815253.8

(51) Int. Cl. A61M 11/00; A61M 15/00; G16H 20/13; B05B 17/00

(52) U.S. Cl. CPC A61M 11/005; A61M 15/0085; A61M 15/0021; A61M 15/009; G16H 20/13; A61M 2205/3553; A61M 2205/3327; A61M 2205/3303; A61M 2205/583; A61M 2205/581; B05B 17/0646

(57) ABSTRACT

A vibrating mesh nebuliser. The nebuliser (100, 400, 500) comprises a cartridge section (110, 420, 520) comprising a reservoir (180) containing a liquid to be nebulised (190), a mouthpiece comprising a conduit (160), wherein a flow path (210) is defined between the reservoir (180) and the conduit (160) and whereby a user may inhale nebulised liquid from the mouthpiece through the conduit (160). The nebuliser further comprises a body section (120, 410, 510), releasably connectable to the cartridge section (110, 420, 520) and a mesh section (530) comprising a vibratable mesh (250, 540), the vibratable mesh (250, 540) being disposable in the flow path (210), so as to nebulise liquid drawn from the reservoir to the conduit in the mouthpiece. Also provided are an apparatus and method for monitoring one or more physiological responses to material self-administered by a user.

### FIELD OF THE INVENTION

The present inventive concept relates to a, delivery device dosing regulator and recommendations engine and related aspects. In a broad sense, the inventive concept includes interacting aspects which form an “ecosystem” of inter-relating apparatus, methods, etc.

The inventive concept relates to the dosing of self-administered material by human users.

### SUMMARY OF INVENTION

A first aspect of the present inventive concept provides a vibrating mesh nebuliser.

A second aspect of the present inventive concept provides apparatus for monitoring one or more physiological responses to material self-administered by a user.

A third aspect of the present inventive concept provides a method for monitoring one or more physiological responses of a user.

A fourth aspect of the present inventive concept provides a kit comprising a plurality of delivery modules each comprising a known composition of one or more active ingredients.

The first aspect of the present inventive concept provides a vibrating mesh nebuliser comprising: i) a cartridge section comprising: a reservoir containing a liquid to be nebulised; a mouthpiece comprising a conduit, wherein a flow path is defined between the reservoir and the conduit and whereby a user may inhale nebulised liquid from the mouthpiece through the conduit; ii) a body section releasably connectable to the cartridge section; iii) a mesh section comprising a vibratable mesh, the vibratable mesh being disposable in the flow path between the conduit and the reservoir, so as to nebulise liquid drawn from the reservoir to the conduit in the mouthpiece. Vibrating mesh nebulisers comprise a mesh having a series of apertures extending therethrough. An aerosol is generated when the liquid to be nebulised is shaken through the apertures when the mesh oscillates.

The cartridge section may be a closed system and provided with the liquid to be nebulised. Once used, the cartridge can be discarded and replaced with a new cartridge. That is, the cartridge section may be a consumable item whereby a user retains the body section and selectively connects a cartridge section to the body section for use. The user may have a range of different cartridges containing different liquids (as described in relation to the fourth aspect of this invention) to be nebulised and may choose one that has been selected by the present method described below.

The mesh section may be integral with the cartridge component or the control component or may be releasably attachable to the cartridge component and/or the body section. If the mesh section is releasably attachable from the cartridge component, or is integral with the body section, then it could be readily cleaned by a user, and also be retained for further use, and as such not be part of a consumable cartridge section. If the mesh section is integral with the cartridge section, it will ensure that that the reservoir and flow path are an entirely closed system.

The mesh section may further include a piezoelectric arranged to oscillate the vibratable mesh, and wherein the nebuliser may further comprise a power supply to selectively power the piezoelectric.

The flow path may further include an air inlet downstream of the mesh section so that air may mix with the nebulised liquid during use. The flow path may further comprise a flow sensor, the flow sensor arranged to detect a user drawing fluid through the mouthpiece during use and to actuate the vibrating mesh when fluid flow is detected. The flow sensor can ensure that liquid is only nebulised when a user inhales from the mouthpiece, to control the amount of liquid dispensed.

The nebuliser may further comprise a valve disposed in the flow path upstream of the mesh section, the valve being biased in a closed position when the cartridge section is disconnected from the body section so as to prevent liquid in the reservoir flowing to the vibratable mesh. The valve may be actuated to an open position whereby liquid in the reservoir is flowable to the vibratable mesh when the body section is connected to the body section. The valve prevents uncontrolled loss of liquid from the reservoir. The valve may be mechanically actuated when the cartridge section and body section are interconnected or may be electronically actuated.

The vibrating mesh nebuliser may further comprise i) at least one sensor to detect one or more of the following: use of the nebuliser; average breath pressure; average breath volume; average breath time; number of breaths; ii) a processing resource in communication with the sensor(s) to process data from the sensor(s) to calculate if the amount of liquid drawn reaches a pre-set threshold dose iii) feedback means communicated from the processing resource to: a) notify a user that they have received the threshold dosage and/or b) actuate cut-off means to deactivate the nebuliser. The at least one sensor may be a flow sensor.

The cartridge section may comprise a single unit comprising a single liquid to be nebulised in a single reservoir. In alternative embodiments, a modular system may be provided whereby a plurality of individual cartridges comprising different liquids to be nebulised are combined into a single cartridge section, so that a plurality of liquids are nebulised concurrently. If multiple cartridges are provided, then the respective cartridges could contain different liquids, or the same liquid but at different concentrations. One cartridge could contain an active ingredient, and another contain water to dilute the active ingredient during use. In some instances, the plurality of cartridges could be used concurrently, or switch means could be provided whereby a user could switch between cartridges as desired. A switch could also be automatic, for example to switch from one cartridge to another depending on input from data communication means, as described below, or if one of the cartridges becomes empty.

In embodiments where there is a plurality of cartridges, the cartridges could be in communication with a single mesh section comprising a single vibratable mesh via a flow path. Alternatively, the mesh section could comprise a plurality of vibratable meshes, individually in communication with a single cartridge via separate flow paths. If multiple vibratable meshes are provided, they could comprise different aperture sizes to create different sizes of droplet. This may be preferred if different ingredients are intended to be inhaled to different parts of the body. For example, some liquids may be favoured to be transported to the lungs, in which case smaller aperture sizes that produce droplets of <5 μm will be preferred. Apertures that produce larger droplets of <10 μm would be preferred if the liquid to be nebulised is intended for absorption in the mouth, or where the liquid is flavoured. Apertures that produce droplets of 5-10 μm will also enter the airways. Vibratable meshes that have a range of aperture sizes to produce a range of droplet sizes could also be provided.

The body section may further comprise data communication means to communicate with apparatus as described above. The data communication means could comprise any known means understood in the art, such as Wi-Fi, IR or wired transmission. The nebuliser can then communicate with the apparatus, which could include an app on a mobile communication device. A user can be advised of the correct cartridge to use by the app, which would also communicate with the nebuliser. When a cartridge section is applied to the body section, the nebuliser could include means, such as RFID tag recognition, to identify whether the cartridge is correct or not, and feedback to the user, via the app, if the cartridge section is not the one recommended. If an incorrect cartridge is connected, the data communication means could prevent the nebuliser form operating, for example by preventing power from being transmitted to the vibratable mesh.

Further information, such an inventory information, could be communicated to a user, and perhaps so that further cartridge sections are automatically reordered.

The body section may also include sensing means such as a heart rate monitor or means to read electrodermal activity. Such readings can be fed to the communication means and communicated to an external data source as herein described. The sensing means could also detect galvanic skin response or heart rate variability, or other biometric measurements.

According to the second aspect there is provided apparatus for monitoring one or more physiological responses to material self-administered by a user, comprising at least one data collection means, a data processing unit and an output.

Suitable data collection means include: a data input device, such as a keyboard or touch screen or the like; a device adapted to measure a physiological factor, such as heart rate, blood pressure monitor or the like; a dose measuring and/or reporting device; a biometric measurement device.

The data processing unit may be provided with a connection to a communications network.

The apparatus may further comprise a data storage unit.

The third aspect of the present inventive concept provides a method for monitoring one or more physiological responses of a user, comprising the steps of: a) receiving physiological data from a data collection means; and/or receiving goal data from a data collection means; and/or receiving administration data from a data collection means; b) processing physiological, goal and administration data with a data processing unit to provide processed data; c) performing an action.

A user can provide information about their current physiological state, by way of one or more measuring and/or monitoring device, or by entering information into a data input device, or by a combination thereof. The user can also provide administration data—i.e. information about material which has been self-administered, if any, and when material was self-administered. In other instances, material may not be self-administered, for example it might be administered by a smart patch, but the doses can nonetheless be monitored.

The user can also provide goal information. Goal information may include specific physiological goals, such as a target heart rate or target blood pressure or the like. Alternatively, goal information may include more generalised or holistic parameters such as a “mood”, “emotion” or “feeling”, which might be “calm”, “relaxed”, “sociable” or “sleepy”.

Physiological data, goal data and/or administration data may be provided by the user by way of a questionnaire. Alternatively, or in addition, such data may be gleaned from application interfaces, or databases, or the like.

The method may further comprise a step of providing a recommendation, based on either data processed by the data processing unit or pre-provided data, or a combination thereof.

A recommendation may include, for example, a dose of self-administered material. Thus, a user may obtain guidance of a dose of self-administered material in order to achieve a desired physiological goal.

The achievement of a physiological goal is likely to be affected by external parameters which are not directly linked to the self-administered dose. These external parameters and data may be predicted and perhaps therefore taken into account by the system, or they may not have been predicted and perhaps therefore be reacted to by the system or by the intervention of the user. For example, weather conditions, “jet lag”, work schedules, other self-administered materials in the body, diet etc. are likely to affect the effect on a user of a particular dose. Furthermore, other factors based on the pre-existing physiology of the user may affect the effect on the user: for example, a self-administered dose may affect different users depending on hormone levels, genetic traits, and the like.

The method may therefore further comprise a step of receiving external data relating to one or more external parameters from a data collection means. External data may be provided by the user by way of a questionnaire. Alternatively, or in addition, external data may be gleaned from application interfaces, or databases, or the like.

The method may comprise a step of establishing a baseline physiological state. For example, physiological data may be collected when the user has not administered any material in the recent past.

The method may thus compare a user's baseline physiological state with a physiological state once material has been administered. Using the physiological data and the administration data the method can establish the effect of the administered material on the user's physiological state.

The method may further comprise storing processed data in a data storage unit. The method may be repeated over time. Thus, the method may build up a set of data points. Such a set may thus record a user's response to administered material

Using a set of data points, the method may be adapted to predict how a user is likely to respond to administered material. Thus, for a given goal the method may recommend material for a user to administer in order to achieve such a goal.

The method may be iterative. Thus, if a recommended material results in a physiological response which is different from that which was expected the set of data may be refined so that a subsequent recommendation may be more accurate.

The method may take into account other data, such as external parameters, pre-existing physiology, etc. in order to refine a prediction.

The method may further comprise a step of anonymising processed data to provide anonymised data points. The method may further comprise a step of transmitting anonymised data points to a remote data store via a communications network. The method may further comprise a step of receiving anonymised data points from a remote data store via a communications network.

Thus, data points generated by more than one user may be aggregated to predict more accurately a user's likely physiological response to an administered material. Thus, the method may provide a more useful recommendation.

Data points may be collated from users of the present inventive concept. Alternatively, or in addition they may be derived from extant data sets collated by other means. For example, there may be extant data sets which correlate moods or other physiological states with physiological data. Thus, extant data sets may be able to describe commonplace states such as “fear”, “stress” and the like in terms useable by the present inventive concept.

Anonymised data may be categorised by external parameters and/or administration data, etc. Thus, to provide a suitable prediction and/or recommendation the method may weight available anonymised data according to external parameters and/or administration data which corresponds most similarly to that of the user.

Data may be stored in a data vault. An individual user's personal data could be stored in a personal data vault. Thus, a user could determine what other party or parties could have access to his or her personal data or part thereof.

In the description of the inventive concept so far, reference has been made to material which is administered. Material may comprise a single active ingredient or a combination of active ingredients in varying proportions. Material may comprise non-active ingredients too, such as aromas or flavours, which may effect aesthetic or psychological effects.

The method may further comprise a step of providing graphics to a user. The method may comprise a step of providing music to a user. The method may comprise a step of providing music recommendations to a user. These steps may assist the user in achieve a goal, for example a particular “mood”.

The fourth aspect of the present inventive concept provides a kit comprising a plurality of delivery modules each comprising a known composition of one or more active ingredients.

A delivery module may comprise a quantity of a single substantially pure active ingredient. Alternatively, a delivery module may comprise a mixture of different active ingredients in known proportions.

Thus, the kit comprises a known set of active ingredients. Quantities of different active ingredients may be selected by selecting a quantity from one or more delivery module.

If a kit provides a range of active ingredients, for example by providing several delivery modules with differing combinations of different active ingredients, then a range of combinations of quantities of active ingredients may be selected. Extant combinations may therefore be made available.

Each delivery module may comprise a composition surrounded by a vessel. The vessel may comprise a delivery means. For example, the vessel may be a bottle or the like and the delivery means may be a pump or spray pump or the like, a pressurised metred dose inhaler. The vessel may be a cartridge section as described in connection with the vibrating mesh nebuliser as herein described. Alternatively, the vessel may be of food grade material. A food grade material vessel may comprise soluble or frangible portions, or a combination thereof.

Information relating to the known set of active ingredients of the kit can be provided to the method as herein described. Thus, administration data can be provided if the quantities administered from the kit are known. Furthermore, a recommendation may comprise a combination of quantities of contents of one or more delivery modules.

The present inventive concept has been described hereinabove without specifying particular active ingredients. The aspects of the inventive concept can be applied to a wide range of delivery means and methods. However, it is envisaged that the inventive concept will be most applicable to orally administered material.

The present inventive concept is described herein with human users in mind, the inventive concept may be applicable without significant modification to animal users.

### DETAILED DESCRIPTION OF THE INVENTION

Exemplary embodiments of aspects of the present inventive concept will now be described in detail, wherein:

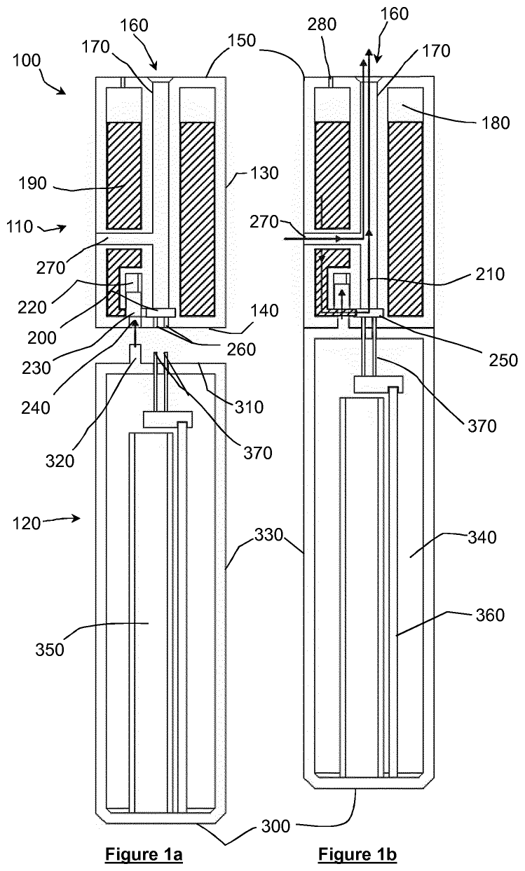

FIGS. 1a and 1b shows a cross-section of a vibrating mesh nebuliser in a connected and disconnected configuration respectively.

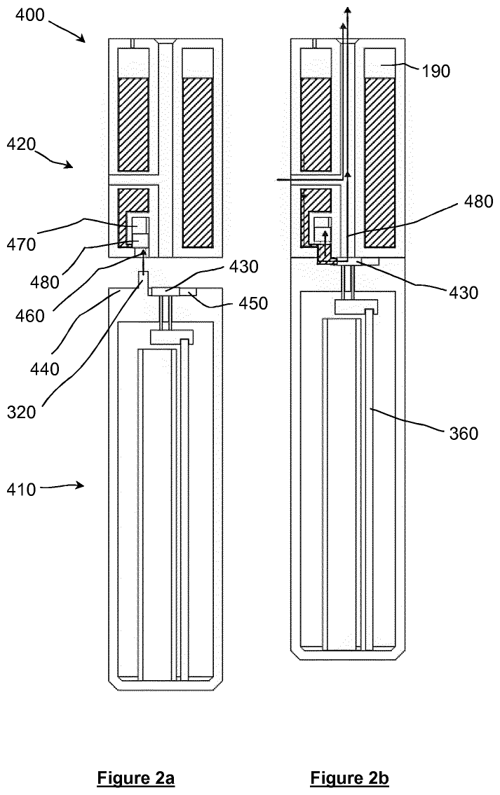

FIGS. 2a and 2b show a cross-section of an alternative nebuliser in a connected and disconnected configuration respectively.

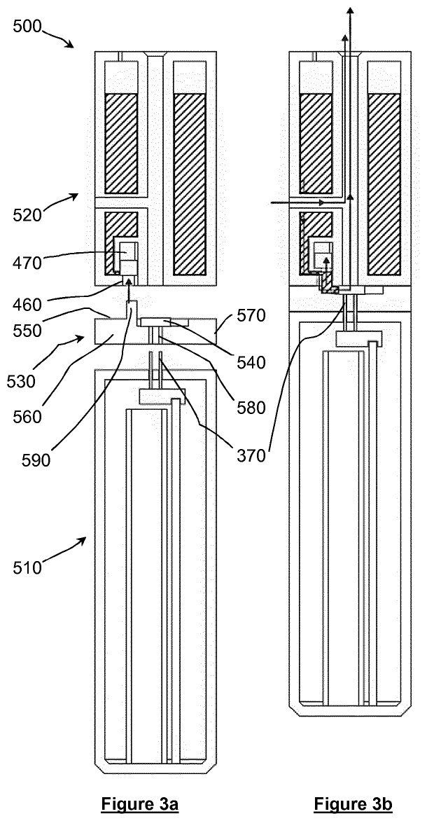

FIGS. 3a and 3b show a cross-section of a further alternative nebuliser in a connected and disconnected configuration respectively.

Turning now to FIGS. 1a and 1b, there is shown a first embodiment of vibrating mesh nebuliser, generally indicated 100. The nebuliser 100 comprises a cartridge section generally indicated 110, and a body section generally indicated 120. Cartridge section 110 comprises a continuous side wall 130 and a generally circular base 140 and an upper surface 150. The side wall 130 and upper surface define a mouthpiece. Extending down from the centre of the upper surface 150 is an outlet 160 having an outer wall 170. The cartridge section 110 is hollow and a reservoir 180 is defined within the side wall 130, the upper surface 150, the base 140 and the exterior of the outer wall 170 of the outlet 160. Within the reservoir 180, shown in cross hatching, is a liquid to be nebulised 190. The reservoir 180 is in fluidic communication with the outlet 160 via an aperture 200 in the outer wall of the outlet so as to define a flow path, as indicated by arrow 210 in FIG. 1b. Within flow path 210 is a gate valve 220 which is biased to a closed position, as shown in FIG. 1a whereby the gate 230 blocks the flow path 210 and prevents the flow of liquid 190 from the reservoir 180 to the outlet 160.

As will be described in more detail later, base 140 comprises an aperture 240 extending therethrough to the gate 230 when it is in the closed position.

Further disposed across the flow path is a vibratable mesh assembly, generally indicated 250. The mesh assembly 250 comprises a vibratable mesh having a series of apertures extending therethrough and a piezoelectric device. Disposed in the base 140 beneath the mesh assembly 250 are twin electrode apertures 260.

An air inlet 270 extends through the side wall 130 and through the outer wall of the outlet 170 to the outlet 160. Air inlet 270 further comprises a flow sensor (not shown) which detects air flow in the air inlet 270.

A narrow venting channel 280 extends through the wall of the upper surface 150 to permit air to enter the reservoir 180, but prevents liquid from exiting the reservoir 180.

The body section 120 comprises a generally circular base 300 and planar upper surface 310, the upper surface substantially mirroring the size and shape of the base 140 of the cartridge section 110, and a continuous side wall 330 extending between the base 300 and upper surface 310 of the body section 120. The side wall 330, base 300 and upper section 310 define a hollow interior 340. The body section 120 is releasably connectable to the cartridge section. Extending from the upper surface 310 is a projection 320 sized and positioned to be inserted into the aperture 240 in the base 140 of the cartridge section 110. When the cartridge section 110 is connected to the body section 120, and the projection 320 is inserted into the aperture 240, the projection 320 pushes up gate 230 to actuate gate valve 220 to an open position, so as to open flow path 210 and permit the flow of liquid 190 from the reservoir 180 to the vibratable mesh assembly 250.

Disposed within the hollow interior 340 is a power source 350 in the form of rechargeable batteries, and a processing resource 360 on a printed circuit board, the processing resource containing data communication means.

The extending through the upper surface 310 of the body section 120 from the interior 340 are electrodes 370, powered by the power source 350. As shown in FIG. 1b, when the cartridge section 110 is connected to the body section 120, the electrodes 370 extend through the twin electrode apertures 260 to provide power from the power source 350 to the piezoelectric in the vibratable mesh assembly 250.

Although not shown here, the body section 120 may comprise a door through which batteries may be removed and replaced, or alternatively the body section 120 may comprise a charging port.

In use, a user attaches a cartridge section containing the desired liquid to be nebulised 190 to a body section 130. The processing resource 360 identifies through a sensor, not shown, that the cartridge section 110 being attached is the correct one and indicates to the user if the cartridge section is correct or not. The cartridge section 110 may have a barcode or RFID tag that permits identification. In connecting the cartridge section to the body section, the projection 320 is inserted into aperture 240 and pushes up the gate 230 in gate valve 220 to actuate the valve 220 to an open position, thus opening the flow path 210 as shown in FIG. 1b. Furthermore, electrodes 370 are inserted into the twin electrode apertures 260 to provide power to the piezoelectric in the vibratable mesh assembly 250. The vibrating mesh nebuliser 100 is thus activated and ready for use.

To receive a dose of nebulised liquid, a user places the mouthpiece into their mouth and inhales. The negative air pressure draws liquid 190 from the reservoir 180 and along the flow path 210 past open gate valve 220 and to a surface of the mesh of the vibratable mesh assembly 250. As the user inhales, air is drawn in through the air inlet 270 to the outlet 170. As air passes the flow sensor 280, a signal is sent via the processing resource to actuate the vibratable mesh assembly 250. The vibratable mesh assembly 250 oscillates, causing the liquid contacting the surface of the mesh to be shaken through the apertures thereof. The liquid 190 is shaken into small droplets that then mix with the air from the air inlet 270 to form an aerosol. The aerosol is then inhaled by the user.

As liquid 190 drains from the reservoir 180, air enters the reservoir 180 through the venting channel 280 to prevent a vacuum from causing liquid flow to cease.

The flow sensor in air inlet 270 further detects at least one of the following parameters: average breath pressure, average breath volume, average breath time and number of breaths. When a threshold value for one of more of the parameters has been met, the processing resource issues a signal to stop the nebuliser, for example by causing power to the piezoelectric to be cut.

The cartridge section 110 can then be removed from the body section 120 and either discarded or saved for later use.

FIGS. 2a and 2b show an alternative embodiment of nebuliser, generally indicated 400, comprising a body section 410 and a cartridge section 420. Like reference numerals will be used where aspects of this embodiment are the same as those shown in FIGS. 1a and 1 b. The significant difference between the embodiment of FIGS. 1a and 1 b, and 2a and 2b is that the mesh assembly 430 of FIGS. 2a and 2b is integral with the body section 410. This is opposed to the embodiment of nebuliser 100 shown in FIGS. 1a and 1b wherein the mesh assembly 250 is integral with the cartridge section 110 and not the body section 120.

The body section 410 comprises a generally circular upper surface 440 in which the mesh assembly 430 is disposed. The mesh assembly 430 is hard-wired to a processing resource 360. The upper surface 440 further includes a depression 450.

The cartridge section 420 includes a circular base 490 in which is disposed gate valve 470 which comprises gate 480 and aperture 460. Aperture 460 is sized and located so as to receive a projection 320 on the upper surface 440 of the body section 410. The aperture 460 is wider than projection 320 so as to form part of a flow path as indicated by arrow 480. Flow path 480 extends from reservoir 190 past gate valve 470 (when open) and past projection 320 to depression 450 and then to the mesh assembly 430, and then from the mesh assembly 430 to outlet 160.

Nebuliser 400 otherwise operates in an identical way to nebuliser 100.

FIGS. 3a and 3b show a third embodiment of nebuliser, generally indicated 500, comprising a body section 510 and a cartridge section 520 and a separate mesh section 530. Like reference numerals will be used where aspects of this embodiment are the same as those shown in FIGS. 1a and 1b and 2a and 2b. The mesh section 530 is removably connectable to the body section 510 and the cartridge section 520.

Cartridge section 520 is essentially the same as the cartridge section 4200 as described in connection with FIGS. 2a and 2b, and like Figure numbers will be used herein.

Body section 510 is essentially the same as the body section 120 as described in connection with FIGS. 1a and 1b, and like Figure numbers will be used herein.

The mesh section 530 comprises a vibratable mesh assembly, generally indicated 540, and as previously described in relation to the embodiments of FIGS. 1a, 1b and 2a, 2b. The mesh section 530 comprises an upper surface 550, a base 560 and a continuous side wall 570. The mesh section 530 is releasably connectable to the body section 510, and the base 560 of the mesh section 530 substantially mirrors the size and shape of the upper surface 310 of the body section 510.

Disposed in the base 560 beneath the mesh assembly 540 are twin electrode apertures 580 extending to the mesh assembly 540 for connection of the electrodes 370 of the body section 510 to provide power to the mesh assembly 540. Although not shown, further means to connect the mesh section 530 to the body section 510 may be provided, such as clip means or interlocking walls.

The mesh section 530 is releasably connectable to the cartridge section 520. The upper surface 550 of the mesh section 530 substantially mirrors the size and shape of the base 490 of the cartridge section 420.

Extending from the upper surface 550 of the mesh section 530 is a projection 590 sized and positioned to be inserted into the aperture 460 in the base 490 of the cartridge section 520. When the cartridge section 520 is connected to the mesh section 530, and the projection 590 is inserted into the aperture 460, gate valve 470 is actuated as previously described. Although not shown, further means to connect the mesh section 530 to the cartridge section 520 may be provided, such as clip means or interlocking walls.

After use, the mesh section 530 can readily be disassembled from the body section 510 and the cartridge section 520 for cleaning.

Nebuliser 500 otherwise operates in an identical way to nebuliser 100 and 400.

The following embodiments are described in respect of a group of active ingredients which are commonly described under an umbrella term “cannabis”. However, the skilled reader will appreciate that the inventive concept is applicable to many other active ingredients with little modification.

There are over 113 different known cannabinoids and a wide variety of terpenes and terpenoids (approximately 140 thereof) and flavonoids (about 20 thereof) present in cannabis, and more are being discovered all the time. These are active ingredients which are key to the experience of using cannabis. Whilst CBD and THC are the most well-known cannabinoids, there are other such as CBN, CBG, CBC, CBE, CBL, CBT, THCA, THCV, CBDV, CBGV, CBGM to name but a few, and the impact of particularly the terpenes and terpenoids can have a multiplying effect (or synergistic effect) on these cannabinoids. A full spectrum product compared to simple isolates is very different in terms of key ingredients and one full spectrum product compared to another will be very different even if key cannabinoids are present in the same quantity/ratio (i.e. 20:1 CBD:THC). Clearly without knowing these details, two apparent similar products based upon a simple ratio of THC to CBD can have wildly different effects on consumers depending upon the make up of other cannabinoids, terpenes/terpenoids and flavonoids, leading to confusion. The present inventive concept aims to provide a consistent and personalised experience meeting the needs of consumers at particular times of the day or when particular events happen, by learning about the particular blend that would suit an individual to meet the desired end state. The particular combination of active ingredients can be important, as some combinations may have a synergistic effect, whereas other combinations could have an antagonistic effect. The ratios of particular active ingredients is also important.

The present inventive concept may use biometric feedback in a closed loop to measure the impact of different blends on an individual (of cannabinoids and terpenes amongst others). It may also become more “intelligent” by learning from population profiles and optimum blends and establishing and/or predicting physiological impact from other information prior to sampling, by collating anonymised data sets.

In use, a questionnaire will be filled in initially (via a smart phone app, for example), to determine a current perceived state and also goals in such areas as stress/anxiety, calm periods, focus (or distracted periods), sleep (quality/efficiency/cycles/durations), cognitive state, energy levels, diet suppression (for weight loss), diet activation (for weight gain), pain, sexual arousal etc. In addition, a biometric wearable maybe included as part of a product package to measure the above states and compare to perceived and required goals and assess overall emotional wellbeing. Physiological measurements could include heart rate, heart rate variation, respiration, GSR, voice analysis, facial analysis, movement etc. which would be triangulated to determine likely states of sleep, focus etc. Note, it is also possible to take data from other existing wearables made by other manufactures. Additionally, factors such as gender and hence production of testosterone or oestrogen and ovulation may be taken into account, as these factors may have an impact on the starting point and desired state of the individual.

Current and target states may also be affected by numerous factors such as travel patterns, including commuting, flying (causing jetlag) or weekend or holiday periods, all which will also have an impact (which may vary by day, week or month) as well as an individual's diet, alcohol consumption or other stimulants such as caffeine or nicotine and diary patterns, including the density of the diary and the types of meetings (who is attending, whether it be a key work related meeting or a social gathering for instance). All of this will need to be taken into account to determine an accurate baseline and to predict what state a consumer would move to if left alone or how to move the consumer to the desired state. These may be entered via a self-reporting method, but integration with other biometric data, such as predicting when a coffee or alcohol has been taken from the readings, is also possible.

It is anticipated that there will be a range of products available to the consumer to manage goals. These products may be provided as a kit or as a delivery module. Products may include, for example, one that is to be taken in the morning, one late morning or after lunch, one to be taken early evening, one before going to sleep (or as required) and recommended doses given. It is possible that some of these could be identical blends or unique. Additionally, there will be some “break glass to open” products, for use such as a particularly stressful occasion (when calming may be needed—blended to that individual) or when particular focus is required as example). Thus, each consumer will have a personalised set of blends that they will use during the day/week. Note, it is anticipated that these will be sublingual sprays or drops but may also be (or in addition to) a set of vaporisers (similar to Dosist™ pens).

One method of optimising the blends for individuals, may be to take the calculated baseline, and send out an initial tailored set of starter blends to each consumer, in small quantities whereby the user will be instructed to take at different times of the day and at different quantities/doses. Depending on the users feeling and biometric data, it will be possible to triangulate this data and revise the required blend set (options) for the consumer and required dosage (to be given to the consumer in the next set of products). As the consumer uses the products and the feedback loop is measured, then iteratively the optimum blends will be developed until a generally steady state is achieved. Note, the very fact of optimising the blends may move the consumer to say a more relaxed or more efficient sleep state, requiring further optimisation (i.e. lower dosing or different composition). Equally for particular events, such as stressful presentations or between seasons, the recommendations may change or evolve. In other embodiments, such a feedback mechanism may be disabled or may not exist at all.

As more data is established across multiple users, predictive starting points base on factors such as genetics, gender and other factors may make the starting point more accurate.

In order to provide precision blends a variety of methods will be used. The key method would be to do a full analysis of each strain/batch/harvest and determine the composition. Based on the number of different strains/harvest available and compositions, the algorithm will design a blend recipe to meet the target blend, in much the same way as tea blenders would take a variety of leaves to make a consistent blend, using different quantities. By using this method, it is more likely to retain the beneficial 30 properties of cannabis than by separating and recombining isolates. This target blend can also be analysed to ensure it is within tolerance on key components (cannabinoids, terpenes, terpenoids, flavonoids and any other ingredients/flavourings required). Another method would be to use isolates and recombine them in the right quantities to meet the target blends, albeit it may be difficult to recombine some of the important elements, but it may be possible as more optimisation and elimination of unnecessary ingredients is established.

It is anticipated that preferred delivery modules will be nebulisers, sprays, droppers or vaporisers. Sublingual sprays may be preferred, as there is no heating of the composition required and thus less risk of degradation thereof. Additionally, a spray may be more acceptable to consumers who have never smoked, as they will not need to inhale vapour into their lungs. Whilst the onset period is longer for sprays than for vaping (10-15 mins with a spray compared to almost instantaneous for vaping), the delivery method may be more acceptable. However, it is anticipated that both sprays and vaporisers will be available and even for the spray kit, a vaporiser may be required for instant results such as when someone is very anxious, so a mixed kit may be appropriate.

Alternative delivery modules can include edibles such as food or drinks. Bespoke drinks made from different blends of active ingredients could be prepared at a venue such as a public house based on a user's personal data.

For the spray, a number of base oils could be used, including hemp oil, olive oil or the current preferred route which is MCT coconut oil. Alternatively, the spray could be water-based.

There will be a feedback mechanism from the spray, dropper, vaporiser or nebuliser, that will download to the app on the smartphone, or via the wearable. Feedback will include amongst other things, which blend was used, how much was dosed, the date and time. This can then be aligned with the corresponding biometric data at the time or lagged (note, the option to self-report on feelings can also be included). Input to the device can be in the form of recommending when to take it. i.e., a green light could come on when the dosage should be taken (as well as highlighting on the app (or messaging email etc.)) and a number of doses (i.e. 1, 2 or 3 sprays). It may have indicators to help this such as orange after the first spray and red once the desired number of doses has been reached. Alternatively, it could be a simple text or icon that is highlighted. This can also be mimicked on the wearable or/and the app. There would also be an indicator to highlight when the spray, dropper, vaporiser or nebuliser is nearly empty (such as 4-7 days usage left based on recommended dosage) and that could trigger an automatic order for replenishment, whether it is the same blend or a further optimised blend. An express option can be available/predicted if an event precipitates it (faster use than predicted or imminent travel highlighted from the calendar).

On the app, the user would be able to see the usage/dosage history and adherence but would also be able to see on the app your current state (i.e. relaxed) and how it had moved the user (from potentially tense to relaxed). It would also show the user potentially where they will be drifting out on the app (i.e. likely to become more stressed at work or possibly when arriving home) and would show how the desired dosage would move you to a different line.

On the wearable, as well as light/text, haptics could be used to alert the user.

Preferably there would be a digital lock on the device for age verification or misuse. This could be controlled via the app or via the wearable using proximity and using either Bluetooth™ or RFI. Alternatively, this could be unlocked via facial recognition, fingerprint recognition or speech recognition. As there will be quite a lot of data in the feedback loop, it is likely that the wearable will deploy Bluetooth™. The spray could contain a micro controller, a battery, a microswitch (to record pushes on the spray), antennas, memory, dataports and LEDs. Alternatively, the spray could be refillable as opposed to disposable, by having the tech on the spray side and a refillable bottle that gets pushed or screwed into the fitting, possibly with a barcode reader to recognise the blend that has been inserted.

The kit could be carried around in a specially designed pack/pouch. Alternatively, a small kit could be arranged on a lanyard, to be used for commonly used varieties, resembling something like a rounded memory stick but would be ergonomic and could be used like a piece of jewelry. In some preferred embodiments, the kit may comprise two types of dose—for example for morning and evening, or perhaps to elicit different preferred states.

It is anticipated that there will be a number of compositions (20 to 40 for example), each being blends of active ingredients that cover a broad enough range so that an individual selection is close to the desired blend (in much the same way as there are a range of shoe sizes to fit individual feet). This can be thought of as mass customisation. However, in time, it is anticipated that this will move to a more custom blended model, whereby 10×10×10 ingredients (30) for example would give 1000 combinations if the 30 quantities were equal, but in varying the quantities of each on there would be an infinite number of possibilities. The 3 sets of ingredients might revolve around cannabinoids, terpenes/terpenoids and flavonoids+the base (at different concentrations). Ultimately it can move towards more of a precision blended model, with highly personalised blends being made for each individual. Compositions would be available subject to local regulation.

Whilst this inventive concept may be aimed as a personalisation model, the model can also be used to facilitate a social occasion and social roles such as easing introductions, casual conversations or providing a change of pace. Therefore, it is possible to assess the state of the group (or those that have opted in) and its interaction and adjust or recommend a blend for each individual to get them in the same relative state.

Biometric data may be “scraped” from existing biometric wearables or from one or more components of smart mobile phones and/or cameras (including a camera integral with a phone) or from a bespoke device.

A composition could be a specific strain of cannabis, or blend of strains or recombining individual elements thereof.

The method could be implemented largely via application software installed on a smart mobile phone. A wearable device may inform a user of a recommendation via a display.

The composition may comprise an oil-based solution. Alternatively, the composition may comprise a water-based solution. A water-based solution may be advantageous for certain active ingredients because of improved bioavailability thereof in a water-based solution or if the solution is delivered with a nebuliser. Thus, a smaller vessel may be needed for the same quantity of bioavailable active ingredient. The solution may comprise a micro- or nano-emulsion.

Preferred delivery modules may be, for example, sprays, droppers, gel strips, edibles including sweets, patches and tinctures.

One further preferred delivery module is a vibrating mesh nebuliser as shown in FIGS. 1a to 3b.

What is claimed is:

1. A vibrating mesh nebuliser comprising:
i) a cartridge section comprising:
a reservoir containing a liquid to be nebulised;
a mouthpiece comprising a conduit, wherein a flow path is defined between the reservoir and the conduit and whereby a user may inhale nebulised liquid from the mouthpiece through the conduit;
ii) a body section releasably connectable to the cartridge section; and
iii) a mesh section comprising a vibratable mesh, the vibratable mesh being disposable in the flow path between the conduit and the reservoir, so as to nebulise liquid drawn from the reservoir to the conduit in the mouthpiece.

2. A vibrating mesh nebuliser as claimed in claim 1 wherein the mesh section is integral with the cartridge section or the body section or is a releasably attachable to the cartridge component and/or the body section.

3. A vibrating mesh nebuliser as claimed in claim 1, wherein the flow path further includes an air inlet downstream of the mesh section so as to mix air with nebulised liquid during use.

4. A vibrating mesh nebuliser as claimed in claim 1 wherein the body section further comprises data communication means.

5. (canceled)

6. A vibrating mesh nebuliser as claimed in claim 1, wherein the flow path further comprises a flow sensor, the flow sensor arranged to detect a user drawing fluid through the mouthpiece during use and to actuate the vibrating mesh when fluid flow is detected.

7. A vibrating mesh nebuliser as claimed in claim 1, further comprising a valve disposed in the flow path upstream of the mesh section, the valve being biased in a closed position when the cartridge section is disconnected from the body section so as to prevent liquid in the reservoir flowing to the vibratable mesh.

8. A vibrating mesh nebuliser as claimed in claim 7, wherein the valve is actuated to an open position whereby liquid in the reservoir is flowable to the vibratable mesh when the body section is connected to the body section.

9. A vibrating mesh nebuliser as claimed in claim 1, further comprising:
i) at least one sensor to detect one or more of the following: use of the nebuliser; average breath pressure; average breath volume; average breath time; number of breaths;
ii) a processing resource in communication with the sensor(s) to process data from the sensor(s) calculate if the amount of liquid drawn reaches pre-set threshold dose
iii) feedback means communicated to from the processing resource to: a) notify a user that they have received a threshold dosage and/or b) actuate cut-off means to deactivate the nebuliser.

10. Apparatus for monitoring one or more physiological responses to material self-administered by a user, comprising at least one data collection means, a data processing unit and an output.

11. Apparatus as claimed in claim 10, wherein the data collection means are selected from the group consisting of: a data input device; a heart rate monitor; a blood pressure monitor; a dose measuring and/or reporting device; a biometric measurement device; and combinations thereof and wherein the data processing unit is provided with a connection to a communications network.

12. (canceled)

13. (canceled)

14. (canceled)

15. A method for monitoring one or more physiological responses of a user, comprising the steps of:
a) receiving physiological data from a data collection means; and/or receiving goal data from a data collection means; and/or receiving administration data from a data collection means;
b) processing the received data with a data processing unit to provide processed data; and
c) performing an action.

16. A method as claimed in claim 15, wherein the physiological data is entered by way of one or more measuring and/or monitoring device, and/or by entering information into a data input device.

17. A method as claimed in claim 15, wherein goal information is one or more of the following: target heart rate; target blood pressure; a mood; an emotion.

18. (canceled)

19. A method as claimed in claim 15, wherein performing the action is displaying one or more pieces of information on a display unit, providing graphics to a user, providing music to a user, or any combination thereof, wherein the information is a recommendation to the user.

20. (canceled)

21. A method as claimed in claim 19, wherein the recommendation is to take a dose of self-administered material, or wherein the recommendation is a music recommendation, wherein the self-administered material is one or more active ingredient.

22. (canceled)

23. A method as claimed in claim 15, further comprising the step of storing processed data in a data storage unit to create a set of data points; and anonymising processed data to provide anonymised data points, then transmitting anonymized data points to a remote data store via a communications network.

24. (canceled)

25. (canceled)

26. A method as claimed in claim 23, further comprise a step of receiving anonymised data points from a remote data store via a communications network then using data points generated by one or more user to predict a user's likely physiological response to an administered material.

27. (canceled)

29. (canceled)

30. (canceled)

31. (canceled)

32. (canceled)

33. (canceled)

34. (canceled)

35. Apparatus according to claim 10 further comprising a plurality of delivery modules wherein each delivery module comprises a known composition of one or more active ingredients.

36. Apparatus according to claim 35, wherein a delivery module comprises a quantity of a single substantially pure active ingredient or a mixture of different active ingredients in known proportions;

37. Apparatus according to claim 35, further comprising a delivery means to deliver the contents of the vessel to a user wherein the delivery means comprises one of a spray pump, a food grade material, a pressurised metered dose inhaler,
